(19) United States
(12) Patent Application Publication
(10) Pub. No.: US 2025/0057627 A1
(43) Pub. Date: Feb. 20, 2025

(54) DEVICE AND METHOD FOR ILLUMINATING A TREATMENT SITE, IN PARTICULAR IN OPHTHALMOLOGY

(71) Applicant: A.R.C. Laser GmbH, Nürnberg (DE)

(72) Inventor: Reinhardt THYZEL, Eckental (DE)

(21) Appl. No.: 18/723,365

(22) PCT Filed: Dec. 21, 2022
(86) PCT No.: PCT/EP2022/087310

§ 371 Date: Jun. 21, 2024

(30) Foreign Application Priority Data
Dec. 21, 2021 (DE) 10 2021 134 143.4

(51) Int. Cl. A61B 90/30; A61F 9/008

(52) U.S. Cl. CPC A61B 90/30; A61F 9/008; A61B 2090/306

(57) ABSTRACT

A device for illuminating a treatment site, such as a human eye comprises a) an applicator and a b) Illumination light source for generating illuminating light (BL), c) wherein the applicator comprises a hollow needle with a distal end and with a target at the distal end and with an opening at the distal end, d) wherein the applicator has an optical fiber guided in the hollow needle to the distal end with a free end oriented towards the target, e) wherein laser pulses (LP) can be transmitted via the light guide, which emerge from the light guide at the free end and strike the target and generate a plasma (P) in front of a target surface of the target, f) whereby the treatment site to be illuminated is located in the area at the opening and the plasma is intended for direct or indirect treatment at the treatment site.

The invention relates to a device and a method for illuminating a treatment site, in particular a treatment site in a human or animal body, in particular in an eye.

In ophthalmology, treatment instruments or applicators are known in which a plasma is generated by laser pulses (hereinafter also referred to as “laser plasma applicators”). The applicator is designed as a hollow needle to be inserted into the eye. At the needle end of the applicator, a target, usually made of titanium (Ti) or a titanium alloy, is provided in the interior of the needle, and an opening in the needle wall, usually also made of titanium (Ti) or a titanium alloy, is provided in the vicinity of the target. The hollow needle also has a laser light fiber (or: light guiding fiber, optical fiber) in the needle interior with a free end opposite the target for guiding and directing the laser pulses onto the target. The laser pulses are generated by a laser and coupled into the laser light fiber at one end and emerge from the other, free end of the optical fiber and then hit the target. These laser pulses are of such intensity (power) and duration that an optical breakdown is generated in the target material, which leads to the formation of a plasma or plasma cloud in front of the target (laser-induced plasma).

The inside of the needle of the applicator is normally filled with a working fluid such as water or an electrolytic irrigation solution such as BSS, which is aspirated from the outside of the eye through the opening and then through the inside of the needle (aspiration with irrigation by other irrigation instruments) or also supplied inside the needle and guided outwards through the opening of the needle (internal irrigation). This irrigation is necessary to prevent a drop in intraocular pressure due to the penetration of the applicator into the eye.

The sudden or very rapid build-up and expansion of the laser-induced plasma leads to a very rapid and strong increase in temperature and pressure in the working fluid, which in turn creates shock waves (or shock waves, pressure pulses) in the working fluid inside the needle interior in the area of the needle end. These shock waves escape to the outside via the opening in the needle wall and can be used to treat the eye. In other applications, the plasma can also escape directly from the outlet opening and be used for treatment in the eye. In both cases, the opening in the needle wall is then an exit opening for escaping plasma or escaping shock waves. However, it is also possible to suck in tissue through the opening and destroy it with the plasma or possibly also the pressure pulses, or to cut it off piece by piece and suck out the remains through the needle. In this case, the opening is therefore an inlet opening for tissue or material entering.

The target surface of the target on which the laser pulses impinge is generally aligned in its spatial arrangement within the needle of the applicator on the one hand partially in the direction of the exit end of the laser light fiber so that the laser light is incident as steeply as possible and a high radiation density or intensity per area is achieved, and on the other hand partially already aligned in the direction of the opening so that the plasma or the pressure pulses or shock or shock waves have a propagation component in the direction of the exit opening. In other words, the effective target surface hit or irradiated by the laser pulses is directed on average or at least in partial areas towards the outlet opening. Seen from this effective target surface, the free end or exit end of the laser light fiber on the one hand and the exit opening in the needle wall on the other hand are therefore in line of sight in a solid angle range of less than 180°, i.e. a laser light beam emerging from the laser light fiber would be reflected by the target surface without plasma in such a way that at least part of this laser light beam would emerge from the exit opening. This alignment of the target surface towards both the laser light fiber and the exit opening, which is always a compromise, is made possible, for example, by an inclined position of a largely flat target surface or a special convex or stepped shape of the target surface in combination with a corresponding arrangement of the exit opening as well as the free fiber end.

The spatial distance of the free end of the laser light fiber from the target is sufficiently large for the selected laser pulse intensity and laser pulse duration so that the maximum spatial expansion of the plasma cannot reach this end of the laser light fiber and damage the sensitive material of the laser light fiber.

The plasma is generated shortly after the start of a laser pulse by the optical breakdown in the target material and continues for a certain time after the end of a laser pulse, whereby the total plasma duration is usually significantly longer than the laser pulse duration of the laser pulse generating the plasma. The plasma duration of the laser-induced plasma depends on the laser pulse intensity and laser pulse duration of the laser pulse and the target material used, as well as on the pressure or volume flow of the surrounding liquid used.

The time intervals between successive laser pulses are now generally chosen to be greater than the plasma duration, so that the plasma of one laser pulse has collapsed again before the next laser pulse arrives. This is because plasma is no longer permeable to electromagnetic radiation below the plasma frequency at the latest at a penetration depth at which a critical electron density is reached and reflects the electromagnetic radiation, thus acting like a mirror for the light.

The laser light used in the aforementioned applicators is usually from the infrared spectrum close to the visible spectrum, e.g. with a wavelength of 1064 nm for a neodymium YAG laser, and thus from a frequency range below the plasma frequencies of the laser-induced plasmas occurring at the target and would therefore not be able to penetrate the plasma to the target if the next laser pulse were already to emerge from the laser light guide as long as plasma with a correspondingly high electron gas density is still present in front of the target.

If the laser pulse duration of the laser pulses is selected in a range from 1 ns to 10 ns, for example, the plasma duration of the plasma is typically in the range from 50 μs to 200 μs, i.e. at least 5,000 times the laser pulse duration. In order to set the intervals between the laser pulses greater than the plasma duration of 50 μs to 200 μs, a pulse frequency (repetition rate) of the laser pulses of less than 20 Hz for a laser pulse interval of more than a plasma duration of 50 μs and less than 5 Hz for a laser pulse interval of more than a plasma duration of 200 μs is selected or set for a regular sequence of laser pulses and for the aforementioned laser pulse durations of 1 ns to 10 ns.

The most common application of such applicators with laser-induced plasma generation (hereinafter referred to as laser plasma applicators for short) in ophthalmology is
destroying the natural lenses of the eye and replacing them with artificial lenses to correct a cataract or to adjust or correct the optical focal length, for example in the case of myopia or hyperopia. Such laser plasma applicators are known, for example, from U.S. Pat. No. 5,324,282 A with an inclined target surface or U.S. Pat. No. 5,906,611 A with a stepped target surface and exit openings at the end of the needle.

Further applications of such laser plasma applicators in ophthalmology are
the removal of epithelial cells in the treatment of secondary cataracts according to WO 2005/107665 A1 or according to EP 2 683 080 B1 with a target at a closed needle end and a lateral outlet opening in the needle wall,
the removal of melanocytes from the stroma to change the eye color according to WO 2018/069013 A1,
the treatment of the trabecular meshwork with plasma for glaucoma therapy according to EP 3 628 281 A1 with a central opening in the needle end of the applicator and
the removal of vitreous body material in vitrectomy according to EP 3 626 213 A1 with a lateral elongated outlet opening in the needle wall and according to the not yet published DE 10 2020 115 885 with an applicator with a curved needle.

The illumination of the treatment site in the eye for the treating person is provided by external lamps or microscope light.

One object of the invention is to provide a new device and a new method for illuminating a treatment site, in particular a treatment site in a human or animal body, in particular in an eye. In particular, an in situ illumination for the area to be treated in the eye is to be provided.

This problem is solved according to the invention in particular by the subject matter of the independent patent claims 1 and 8. Embodiments and further embodiments result from the dependent patent claims as well as from the following description and the drawings.

The claimable combinations of features and subject-matter according to the invention are not limited to the selected wording and the selected back references of the patent claims. Any feature in the claims, even independently of their back references, may be claimed in any combination with one or more other feature(s) in the claims. In addition, any feature described or disclosed in the specification or drawing may be claimed by itself, independently or divorced from the context in which it appears, alone or in any combination with one or more other features described or disclosed in the claims or in the specification or drawing.

In the following, embodiments according to the invention are described. Reference is also made to the drawings, in which

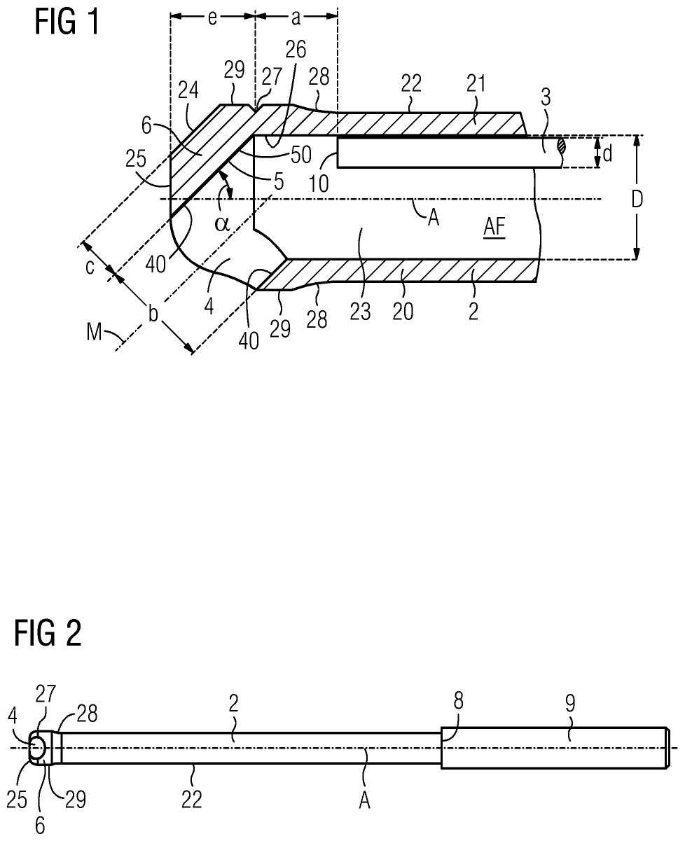

FIG. 1 a hollow needle of an applicator in the front area with its distal end in a longitudinal section,

FIG. 2 the hollow needle according to FIG. 1 in an overall view from below,

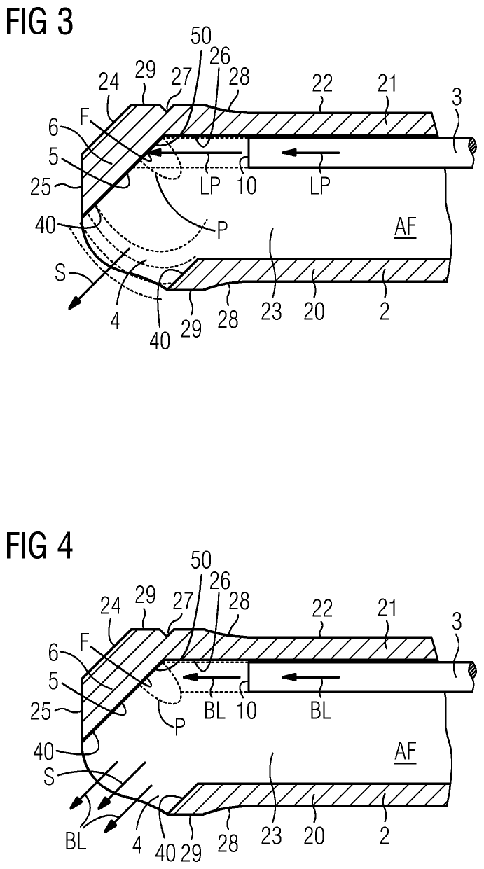

FIG. 3 longitudinal section of the hollow needle according to FIG. 1 in the front area with its distal end during operation of the applicator when generating plasma by means of a laser beam,

FIG. 4 the hollow needle according to FIG. 1 and FIG. 3 in the front area with its distal end when illuminated by means of an illumination light during operation of the applicator in a longitudinal section,

FIG. 5 the hollow needle according to FIG. 4 in an internal view of the distal end,

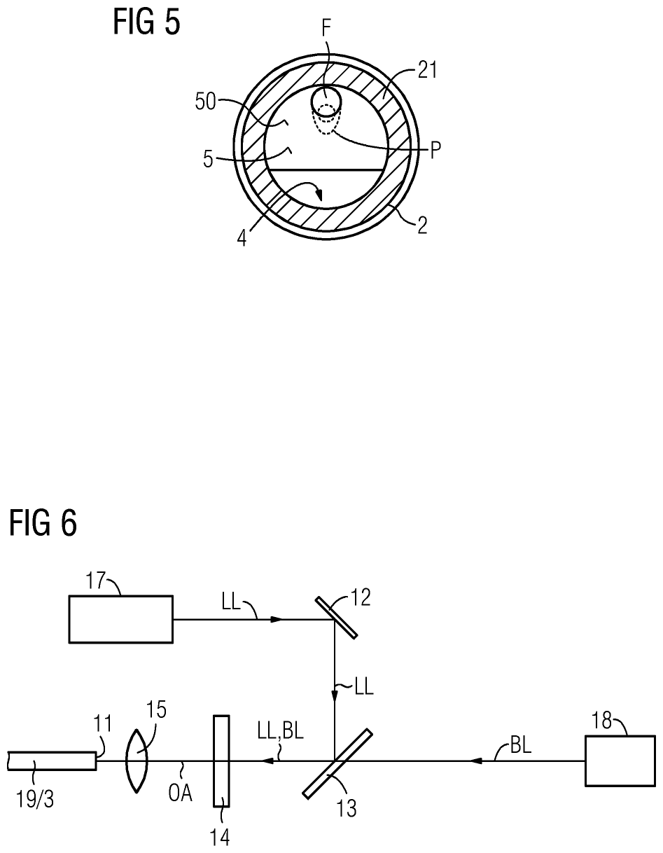

FIG. 6a coupling device of an applicator, in particular according to FIGS. 1 to 4, for coupling a light guide to a laser light source and an illumination light source, and

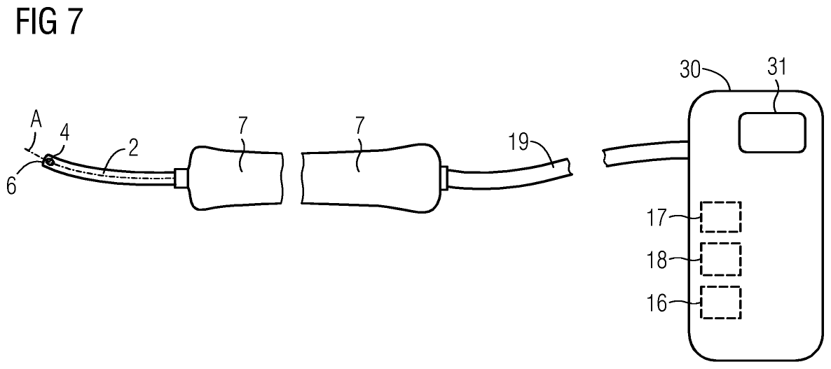

FIG. 7a device for vitrectomy with an applicator and a central unit with laser light source and illumination light source as well as a coupling device

are shown schematically in each case. Unless explicitly described otherwise, identical or functionally identical elements are designated with the same reference signs in the figures. Furthermore, the figures are not necessarily true to scale.

In the embodiment according to FIG. 1 and FIG. 2, an applicator 2 comprises a hollow needle 20, the needle wall 21 of which encloses a cavity or needle interior 23 of the hollow needle 20 and which extends along an applicator axis (or generally an applicator central line if the hollow needle 20 is also curved) A, and a light guide 3, which extends in the needle interior 23 of the hollow needle 20.

An inner needle diameter D of the needle interior 23 is typically selected from a range of 0.1 mm to 0.9 mm or, based on medical terminology, in the range of 20 G to 25 G (G: gauge).

A target 5 with a target surface 50 is arranged at a distal end 6 of the hollow needle 20 in the needle interior 23. A free end 10 with an optical exit surface of the light guide 3 is located opposite the target surface 50 at a distance a. The distance a is in particular between 0.7 mm and 1.2 mm, in particular about 0.9.

The light guide 3 is preferably an optical fiber or optical fiber with a diameter d that is typically selected from a range of 150 μm to 400 μm, in particular 283 μm.

Furthermore, an opening 4 is formed in the needle wall 20 in the region of the distal end 6 with an opening diameter b and a central axis M and an inner opening surface 4 surrounding the opening 4. The inner space of the hollow needle 20 is connected to the outer space through the opening 4. During operation of the applicator during treatment, the outer space is a treatment area in the human or animal body, preferably in an eye, into which the hollow needle 20 has been inserted with its distal end 6. In the illustrated embodiment example, the central axis M of the opening 4 is inclined relative to the applicator axis A by an angle of inclination α which is 45°, but is generally preferably selected from a range of 30° to 60°. The opening diameter b is selected in particular from a range of 0.2 mm to 1.4 mm, in particular approximately 0.7 mm.

The hollow needle 20 and its needle interior 23 can be produced from a solid blank by inserting a special first drill with a drill outer diameter corresponding to the desired needle inner diameter D of the needle interior 23 axially to the applicator axis A, starting from a proximal end 8 opposite the distal end 6, wherein the needle wall 21 remains stationary with a predominantly cylindrical outer surface 22 and wherein drilling the needle wall 21 at the distal end 6 initially produces a conical inner surface which forms the drill tip, for example with an opening angle of 60°.

The opening 4 can now be created by inserting a second drill bit through the needle wall 21 in the area of the distal end 6 in the drilling direction along the center axis M with an outer drill diameter corresponding to the opening diameter b. This subsequent drilling of the opening 4 produces a (further) cylindrical inner surface of the needle wall 21, which continues from the inner opening surface 40 of the opening 4 and a target surface 50 of the target 5, which is then also produced with this second drill and extends on a cylindrical surface around the center axis M with the cylindrical diameter b. The cylindrical target surface 50 of the target is then inclined at the angle of inclination a to the applicator axis A, just like the central axis M. The target surface 50 can also have an additional conical section that remains from the first drilling process or the first drill.

The target 5 is formed as, in FIG. 1 middle, a partial area of the thicker wall region of the needle wall 21 with the axial length e and the greater wall thickness c, which also corresponds to the wall thickness of the target 5, which remains at the distal end 6 of the hollow needle 20 after the two drilling steps. This thicker wall area of the needle wall 21 is bounded on the outside of the needle wall 21, for example, by a bevel 24, which can be conical or in the form of a chamfer, and on the end face by a flat end face 25 directed perpendicular to the applicator axis A.

The greater wall thickness c of the target 5 behind the target surface 50 can, without limiting the generality, be in particular between 0.1 mm and 0.5 mm and serves, among other things, to take account of the target material removal by the laser pulses LP. Outside the target 5 or in the area of the outer surface 22, the needle wall 21 can have a smaller wall thickness, for example around 0.1 mm.

However, it is also possible, in particular by means of other manufacturing steps, and within the scope of the invention, to produce a different shape of the target surface 50, for example at least partially a conical shape, in particular with the applicator axis A as the axis of symmetry, or a flat shape or also a stepped shape or a shape which is concave or convex in longitudinal section.

Furthermore, as shown in FIG. 1 (and FIGS. 3 and 4), the needle wall 21 is enlarged to a larger outer diameter in the region of the distal end 6, generally already in the blank before the needle interior 23 is created in the first drilling step. The cylindrical outer surface 22 of the needle wall 21 with a predetermined outer diameter merges towards the distal end 6 via a transition region 28, for example concavely curved, into a cylindrical outer surface 29 of the needle wall 21 with a predetermined outer diameter which is larger than the outer diameter of the outer surface 22. This can also serve, for example, to protect a so-called sleeve as a seal against forward stripping.

In the axial area of the needle wall 21, in which the second bore for the opening 4 or the target surface 50 merges into the cylindrical inner surface of the needle wall 21 with the inner diameter D, a notch or marking 7 is also provided on the outside of the needle wall 21, which is provided on the opposite side partially from the opening 4 and marks the position of the opening 4.

The operating principle and the function of the applicator according to FIGS. 1 and 2 for laser-induced plasma generation are now explained with reference to FIG. 3. Laser-induced plasma generation is known per se, in particular with the applicators with laser-induced plasma generation mentioned at the beginning.

Laser light (or: laser radiation) is transmitted to the distal end 6 of the hollow needle 20 by means of the light guide 3, usually in the form of laser pulses LP. The laser pulses LP emerge from the free end 8 of the light guide 3 directed towards the target 5 and strike the target surface 50 in a focal area F of the laser pulses LP. The power area densities or energy area densities and the pulse durations of the laser pulses LP are now selected such that an optical breakdown occurs near the surface of the given target material of the target 5 at the target surface 50 and plasma P emerges into the needle interior 23 in front of the target surface 50.

With a diameter d of the light guide 3 of 270 μm, this results in a cross-sectional area at the free end 10 of approximately (0.27 mm)²π=0.23 mm² for calculating the energy area density or power area density.

The pulse durations of the laser pulses LP are typically selected from a range of 1 ns to 10 ns, depending on the application of the applicator 2. The energy per laser pulse LP is preferably set from a range of 2 mJ to 15 mJ. Such laser pulses LP generate plasma P with a plasma duration typically in the range from 50 μs to 200 μs, i.e. at least 5,000 times the laser pulse duration. This means that the state shown in FIG. 3 is only an initial or induction state for a short time, namely the pulse duration of the laser pulse LP, and then a state without laser pulse LP, but with the plasma P, i.e. as in FIG. 3, only without the laser pulse LP, prevails for a much longer time.

The laser light for the laser pulses LP is usually from the infrared spectrum close to the visible spectrum, e.g. with a wavelength of 1064 nm, as supplied by a neodymium YAG laser. Such laser radiation is from a frequency range far below the plasma frequencies of the laser-induced plasmas P occurring at target 5.

Since the built-up plasma P reflects the laser radiation, the time intervals of the laser pulses LP are set to be greater than the plasma duration of the aforementioned 50 μs to 200 μs or a pulse frequency or pulse repetition rate of the laser pulses of less than 20 Hz is set for a laser pulse interval of more than a plasma duration of 50 μs and less than 5 Hz for a laser pulse interval of more than a plasma duration of 200 μs.

During the optical breakdown, the high-power laser pulses LP at the target surface 50 in the irradiated focal area F not only release (quasi) free electrons from the conduction energy band (conduction electrons), but also electrons bound by an avalanche effect from the electron shells of the atoms of the target material structure, thereby additionally ionizing target atoms. The laser pulse energy transmitted during the laser pulse duration is generally only selected to be so high that essentially only electrons and no ionized target atoms are released into the plasma P from the target 5.

The target material of the target 5 is therefore preferably a metal or a metal alloy due to the already existing conduction electrons and high electron density and the optical breakdowns that take place much faster or at much lower laser powers and less material damage compared to dielectrics. A preferred material for the target 5 is titanium (Ti) or a titanium alloy, preferably made of or with titanium (Ti), whereby the titanium can be Ti grade 4, for example. In the illustrated embodiment example, the target 5 is formed in one piece with the needle wall 21, which thus consists of the same material, in particular metal or metal alloy, preferably titanium (Ti) or a titanium alloy.

The plasma P emitted from the target 5 expands and spreads at high speed away from the target surface 50 inwards into the needle interior 23 and assumes approximately the shape of a projectile or dome-shaped and, as seen from the target surface 50, convex plasma cloud, as outlined in FIGS. 3 and 4. This convex or quasi-focused shape of the plasma cloud P, as seen from the focal region F, also results from the convex and quasi-focusing, in particular cylindrical and/or conical, shape of the target surface 50 in the focal region F.

During operation of the applicator 2 in an eye treatment, the needle interior 23 is normally filled with a working or irrigation fluid AF such as water or an electrolytic irrigation solution such as BSS, which is aspirated from the outside of the eye through the opening 4 and the needle interior 23 and is supplied by an irrigation instrument, or is also supplied through the needle interior 23 and exits to the outside through the opening 4. This irrigation is necessary to prevent a drop in intraocular pressure due to the penetration of the applicator into the eye.

This irrigation or working fluid AF now dampens and slows down the expansion of the plasma P inside the needle 23 (“extinguishes” the plasma). At the same time, however, the sudden increase in pressure and the very sharp rise in temperature caused by the laser-induced plasma P leads to the formation of shock waves S (or pressure pulses, shock waves), which propagate in the liquid AF in the needle interior 23 and escape at least partially to the outside through the opening 4. Depending on the application, high pulse rates and thus shock wave rates are also selected and/or the next laser pulses LP can be sent as close as possible to the end of the plasma duration or already during the collapse of the preceding laser pulses LP in order to “maintain” the plasma.

These shock waves S generated by the laser-induced plasma are now usually the actual treatment medium for the treatment.

Preferred applications of the applicator 2 with laser-induced plasma generation and the shock waves generated with it in ophthalmology are the eye lens interventions already mentioned at the beginning, such as the destruction of the natural eye lenses and replacement with artificial lenses to correct a cataract or to adjust or correct the optical focal length, for example in cases of myopia or hyperopia, the removal of epithelial cells in the treatment of secondary cataracts and the removal of melanocytes from the stroma to change the color of the eye.

A further (direct) application of plasma P from applicator 2 is possible when opening the trabecular meshwork for glaucoma therapy and when removing vitreous body material during vitrectomy.

Furthermore, medical applications outside of ophthalmology are also possible, e.g. in lithotripsy to break up gallstones or kidney stones and also applications outside of medicine, e.g. in the fine restoration of works of art and architecture, e.g. frescoes.

Now that the main function of the applicator has been sufficiently described, we will now turn to the new additional function for the applicator according to the invention, namely the integrated lighting.

According to the invention in general and the embodiments shown in FIG. 4 and FIG. 5 in particular, in situ illumination or illumination is provided in the applicator for the location where the treatment takes place. For this purpose, illumination light is sent from the hollow needle to the treatment site through the same light guide through which the laser pulses are sent to generate the plasma.

Experts, including the inventor of the present invention, who has decades of experience in this field, would not have thought that this was physically possible. Illumination light from the light guide follows the same beam path as the laser pulses to the target and thus inevitably encounters the plasma generated by the laser pulses in front of the target surface, which lasts considerably longer. Since the optical frequency spectrum of illumination light, especially white light, is below the plasma frequency, the illumination light is reflected by the plasma as if by a mirror and therefore cannot penetrate the plasma and reach the opening and thus cannot spread to the treatment site.

Nevertheless, technical considerations and tests by the inventor have shown that a considerable proportion of the illumination light from the light guide can still reach the opening in the needle wall and from there to the outside, despite the plasma, if certain technical specifications for the applicator are set and adhered to.

The invention is based on the idea of using the plasma and the inner wall of the hollow needle at the distal end as reflective or mirror surfaces for the illumination light and thus guiding the illumination light outwards through the opening, in particular the opening 4, by means of multiple reflections on the plasma and the inner wall of the needle.

The laser pulses are set with respect to their power density values and pulse durations and the distance a of the free end 10 of the light guide 3 from the target surface 50 is selected large enough so that the plasma generated by the laser pulses expands less far away from the target surface 50 than the distance a or gap between the target surface 50 and the free end 10 of the light guide 3 and thus remains far enough away from the free end 10 of the light guide 3, as shown in FIG. 4. The values given above for the laser pulses LP and the distance a already fulfill these conditions. As a result, the plasma is always at a distance from the free end of the light guide and the proportion of the illumination light BL reflected backwards away from the opening 4 is kept small.

Furthermore, it was observed by measurements that the plasma, starting from the focal area of the laser light from the light guide on the target surface, assumes a convex and elongated expansion or shape as seen from the target surface, as shown schematically in FIGS. 3 and 4. As a result, there is a convex plasma mirror surface in the beam path of the illumination light emerging from the light guide, which fills the entire beam cross-section of the illumination light in the projection, but which also reflects the illumination light BL over a substantial part of the beam cross-section with a component in the direction of the propagation direction of the illumination light as it emerges from the light guide due to its convex elongated shape.

Finally, the inner wall of the hollow needle, i.e. the inner surface 26 of the needle wall 21 and the opening inner surface 40, must be designed to be light-reflecting or specular, at least in the region of the distal end 6, so that the illumination light BL reflected by the plasma P towards the inner surface 26 is reflected again by the inner surface 26 of the needle wall 21 and then either already passes the plasma P on the outside or finally reaches the opening in the needle wall through multiple reflections between the inner surface 26 of the needle wall and the plasma and can exit there.

The mirror surface on the inner surface 26 and 40 (and also 50) of the needle wall 21 (and 6) is preferably very smooth, for example with a roughness depth of less than 10 μm, and is produced in particular by the fine drills rotating at very high speeds of 6000 to 8000 rpm and moving under high feed pressure to produce the needle interior 23 and the opening 4. These multiple reflections towards the opening 4 are supported by the convex, in particular cylindrical, shape of the inner surfaces 26 and 40, which guide or concentrate the illumination light BL towards the opening 4 or its central axis M.

FIG. 4 and FIG. 5 now show very schematically and sketchily the situation with a plasma P still present after the laser pulse LP has been switched off and at the same time the illumination light BL now entering the needle interior 23 from the light guide 3 at the free end 10.

The illumination light BL is light from the visible spectrum with a predetermined or adjustable frequency spectrum, in particular white light with a broad frequency spectrum from the visible range, in particular 400 nm to 800 nm or 420 nm to 780 nm.

Now the illumination light BL, just like the laser light of the laser pulses LP before, is directed towards the focal area F on the target surface 50, but does not reach the target surface 50, but first hits the plasma P in front of the target surface 50. The illumination light BL is now reflected by the plasma P, since the light frequencies of the illumination light BL are below the plasma frequency. After reflection at the plasma P, the illumination light BL then strikes the inner wall, in particular the inner surface 26 or 40 of the needle wall 21 and is reflected inwards there again, then strikes the plasma P again or runs past the plasma P to the opening 4. Due to these multiple reflections, the illumination light BL ultimately reaches the opening 4 despite the presence of the plasma, at which the illumination light BL then emerges, as shown in FIG. 4. The opening 4 is also chosen to be relatively large, which means that more of the multiple reflected illumination light BL can be collected and can emerge from the opening 4.

The proportion of the illumination light reaching the opening 4 through multiple reflections from the free end of the light guide is further enhanced by the comparatively small area of the focal area F and thus of the plasma F compared to the total cross-sectional area D²π of the hollow needle and by the convex design of the inner wall of the needle in relation to the central axis M of the opening 4 and the applicator axis A as described above. The partial orientation of the target surface 50 towards the opening 4 also increases the portion of the illumination light BL that is reflected several times towards the opening 4.

In particular, the illumination light BL can light up continuously or emerge from the light guide 3 when it is switched on by the user, i.e. it can also be switched on during the laser pulses LP.

Illumination directly at the treatment site in the eye has the particular advantage for patients that the comparatively bright and unpleasant microscope light can be dimmed during the procedure.

FIG. 6 now shows a coupling device for coupling a proximal end 11 of the light guide 3 (or an intermediate connecting light guide 19) facing away from or opposite to the distal end 10 with both a laser light source 17, which emits the laser pulses LP, for example an Nd: YAG laser source, and an illumination light source 18, which emits the illumination light BL. The laser pulses LP from the laser light source 17 are fed via a deflecting mirror 12 to a beam combiner 13, which is formed with a partially transparent or IR-reflecting mirror that transmits visible light and is arranged at 45°, and are deflected by this mirror by 90° onto an optical axis OA The illumination light BL from the illumination light source 18 is fed to the beam combiner 13 offset by 90° and passes through it in a straight line and then also runs along the optical axis OA. The laser pulses LP and the illumination light BL are now fed into the end 11 of the light guide 3 or 19 via a shutter 14 and a converging lens 15. The illumination light source 18 can be formed with several diodes, for example RGB, in order to be able to adjust the frequency spectrum or also a color temperature of the illumination light BL.

Instead of a single optical fiber, in embodiments not shown the light guide 3 can also comprise several optical fibers, in particular optical fibers guided next to one another, with the laser light and the illumination light each being guided through one of these several optical fibers. In further embodiments, it is also possible that one or more optical fibers of the light guide are provided for the laser light, and that one or more optical fibers are provided for the illumination light.

In principle, in an alternative embodiment not shown, a light guide or an optical fiber for the illumination light can also end closer to the opening or away from the plasma region, so that the illumination light can pass through the opening to the outside without first encountering the plasma.

An entire ophthalmologic device, in particular for vitrectomy, is shown in FIG. 7. The applicator 2 is designed here as a vitrectome and the hollow needle 20 is curved at least in sections for better access in the eye, in particular in the end section up to the distal end 6, at which the opening 4 is provided. The hollow needle 20 is coupled to a handle part 7 and its light guide 3 is optically coupled to a light guide 19 (a light guide cable), which is optically connected at the other end to a central unit 30. The central unit (or interface) 16 has a laser light source 17 and an illumination light source 18 as well as a coupling device for optically coupling the laser light source 17 and the illumination light source 18 to the light guide 19 and thus to the vitrectome or applicator 2.

Furthermore, a suction unit 16 is provided, in particular in the central unit 30, which is connected to the needle interior 23 of the hollow needle 20 and generates a negative pressure of, for example, 0.2 bar to 0.4 bar under atmospheric pressure for sucking vitreous body material into the needle interior 23 for ablation of the vitreous body by means of the plasma P and for suctioning off the ablated vitreous body pieces. A replacement fluid such as silicone oil can be supplied to the eye to compensate for the ablated vitreous body material.

A corresponding device can also be combined with another applicator, for example an applicator according to FIGS. 1 to 5 and/or be provided for other applications. The device may comprise a control or monitoring device for controlling or monitoring the laser light source and the illumination light source. The control device may comprise one or more electronic circuits and/or processors. The control device, in particular the electronic circuit and/or the processor or processors, is/are set up to control at least the laser light source and the illumination light source for carrying out or performing a treatment.

The device can comprise further components, for example an irrigation device for introducing irrigation or replacement fluid into the eye in addition to or instead of the suction unit.

### LIST OF REFERENCE SYMBOLS

2 Applicator
3 Light guide
4 Opening
5 distal end
7 Handle part
8 proximal end
9 Intermediate part
10 free end
11 End of the light guide
12 Mirror
13 Beam combiner
14 Shutter
15 Lens
16 Extraction unit
17 Laser light source
18 Illumination light source
19 Light guide
20 Hollow needle
21 Needle wall
22 Exterior surface
24 Bevel
25 Front face
26 Inner surface
27 Ring groove
28 Transition area
29 Exterior surface
30 Central unit
31 User interface
40 Opening inner wall
50 Target surface
a Distance light guide to target
b Diameter opening
c Wall thickness
d Diameter of light guide
c axial length
A Applicator axis
BL illuminating light
D Inside diameter of hollow needle
F Focus area
LL Laser light
M Middle axis opening
O Aoptical axis
P Plasma
α Tilt angle

1-10. (canceled)

11. A device for illuminating a treatment site, in particular a treatment site in a human or animal body, in particular in an eye, comprising:
a) an applicator and b) an illumination light source for generating illuminating light,
c) wherein the applicator comprises a hollow needle with a distal end and with a target at the distal end and with an opening at the distal end,
d) wherein the applicator has an optical fiber guided in the hollow needle to the distal end with a free end oriented towards the target,
e) wherein laser pulses are transmittable or transmitted via the light guide, which emerge from the light guide at the free end and strike the target and generate a plasma in front of a target surface of the target,
f) whereby the treatment site to be illuminated is located in the area at the opening and the plasma is intended for direct or indirect treatment at the treatment site,
d) the illumination light source being optically coupled or couplable to the light guide of the applicator in such a way that the illumination light of the illumination light source is transmitted via the light guide and emerges from the light guide at the free end,
e) wherein an inner surface of the hollow needle at the distal end is designed as a mirror surface for the illuminating light, such that the illuminating light is reflected from this mirror surface after emerging from the light guide and at least partially with or after one or more intermediate reflections from the plasma to the opening.

12. The device according to claim 11, wherein the mirror surface is arranged at least around the target and around the opening.

13. The device according to claim 11, further comprising:
a laser light source for generating the laser pulses, and
an optical coupling device for direct or indirect optical coupling of the light guide to the laser light source and the illumination light source.

14. The device according to claim 11, wherein:
the illuminating light is white light and/or is selected from the optical frequency spectrum below the plasma frequency of the plasma; and
the plasma acts as a reflection or mirror surface for the illumination light.

15. The device according to claim 11, wherein the distance of the free end of the light guide from the target surface is selected to be so large that the plasma generated by the laser pulses is spaced from the free end of the light guide.

16. The device according to claim 15, wherein the distance of the free end of the light guide from the target surface corresponds to a value between 0.7 mm and 1.2 mm, in particular approximately 0.9 mm.

17. The device according to claim 11, wherein the opening has a central axis which is inclined relative to an applicator axis of the hollow needle wall by an angle of inclination, and has a cylindrical opening wall.

18. The device according to claim 17, wherein the angle of inclination is selected from a range of 30° to 60°, preferably 45°.

19. The device according to claim 11, wherein:
a needle interior of the hollow needle is produced by means of a first drill inserted along the applicator axis, and
opening is produced by means of a second drill inserted along the center axis, and
smooth mirror surfaces for the illumination light are preferably produced by means of high drill speeds, for example from 6000 to 8000 rpm.

20. A method for illuminating a treatment site, in particular a treatment site in a human or animal body, in particular in an eye, wherein:
a) an applicator which comprises
a1) a hollow needle with a distal end and with a target at the distal end and with an opening at the distal end and
a2) further comprises an optical fiber guided in the hollow needle to the distal end with a free end oriented towards the target,
is used and
b) the opening is located at the distal end of the hollow needle of the applicator at the treatment site to be illuminated,
c) laser pulses from a laser light source are coupled into the light guide and emerge from the light guide at the free end and strike the target and generate a plasma in front of a target surface of the target,
d) illuminating light from an illumination light source is coupled into the light guide and emerges from the light guide at the free end, and
e) the illuminating light, after emerging from the light guide, is reflected from the plasma to the opening and the treatment site at an inner surface of the hollow needle formed as a mirror surface with one or more intermediate reflections.

21. The method according to claim 20, wherein:
the laser pulses have a pulse duration in the range from 1 ns to 10 ns and/or have a pulse energy in the range from 2 mJ to 15 mJ; and/or
the distance between two laser pulses is selected to be greater than the plasma duration of the plasma, the plasma duration being in particular in the range from 50 μs to 200 μs.

22. The method according to claim 20, wherein:
an illumination duration of the illumination light is greater than the duration of a plurality of laser pulses in succession; and/or
the illumination light is coupled in continuously.
